(19) United States
(12) Patent Application Publication
(10) Pub. No.: US 2020/0107751 A1
(43) Pub. Date: Apr. 9, 2020

(54) COMPACT ANTENNA ARRANGEMENT OF RADAR SYSTEM FOR DETECTING INTERNAL ORGAN MOTION

(71) Applicant: Siemens Medical Solutions USA, Inc., Malvern, PA (US)

(72) Inventors: Ahmadreza Ghahremani, Knoxville, TN (US); James J. Hamill, Knoxville, TN (US)

(21) Appl. No.: 16/154,002

(22) Filed: Oct. 8, 2018

(51) Int. Cl. A61B 5/08; G01S 13/42; A61B 5/024; A61B 5/0205; G01S 7/35

(52) U.S. Cl. CPC A61B 5/08; G01S 13/426; G01S 7/352; A61B 5/0205; A61B 5/024

(57) ABSTRACT

A compact radar system for detecting displacement of an internal organ of a patient in a medical scanner. The system includes at least one transmitting antenna and at least one receiving antenna located in a bed arrangement that supports the patient. In particular, the receiving antenna is located a predetermined distance from a patient reference location to enable detection of electromagnetic energy reflected from a region of the internal organ undergoing asymmetric displacement. The system further includes a radar energizing system that energizes the transmitting and receiving antennas wherein the transmitting antenna irradiates a volume of the patient's body that includes the internal organ. In addition, the receiving antenna detects the reflected electromagnetic energy from the region of the internal organ undergoing asymmetric displacement to enable determination of inhalation and exhalation by the patient.

### TECHNICAL FIELD

Aspects of the present invention relate to a compact radar system for detecting displacement of an internal organ of a patient positioned in a medical scanner, and more particularly, to a compact radar system for detecting displacement of an internal organ of a patient in a medical scanner that includes at least one transmitting antenna and at least one receiving antenna wherein the receiving antenna is located a predetermined distance from a patient reference location to enable detection of asymmetric displacement of the internal organ.

### BACKGROUND

Medical imaging techniques such as positron emission tomography (PET), computed tomography (CT), single-photon emission computed tomography (SPECT) and others are used to obtain images of the interior of a patient's body. During a diagnostic scan utilizing such imaging techniques, the patient's respiratory motion can cause undesirable image artifacts, or the incorrect alignment of two modalities due to internal organ movement that occurs during patient respiration.

In order to overcome these disadvantages, conventional imaging systems utilize respiration-correlated gating techniques to obtain a respiration waveform. The waveform is then used to correlate respiration with time so as to provide motion correction of image data. Such systems typically include devices and sensors that are positioned on the patient by a trained operator. For example, a strain gauge or an optical tracker may be attached to a patient to measure chest elevation during respiration. However, the operation and accuracy of such systems is dependent on system setup and operator training. For example, pressure sensors used in some types of systems require adjustment by a trained operator prior to use. Further, the pressure sensors may loosen during a scan and require repositioning by the operator in order to maintain accuracy. In other types of systems that utilize optical detection of skin location, a line of sight path between a target and sensor is required that may be obscured by blankets, bent knees etc. of the patient. Moreover, the systems require substantial setup time and are not user friendly.

Alternatively, a Doppler radar system may be used to detect internal organ movement. Such systems operate within the ultra high frequency (UHF) bandwidth of the electromagnetic spectrum and include a patch antenna that emits electromagnetic (EM) radiation that irradiates a relatively large volume of the patient's body. This creates undesirable reflections of EM radiation from various organs within the body that are not of interest for detecting respiration. In addition, EM radiation may reflect off surfaces located outside of the patient's body, such as a CT gantry surface of an imaging system, wall or other surface. The reflections from organs that are not of interest and from surfaces outside the body result in undesirable noise in the reflected radar signal and a relatively low signal to noise ratio (SNR).

### SUMMARY OF THE INVENTION

A compact radar system is disclosed for detecting displacement of an internal organ of a patient in a medical scanner. The system includes at least one transmitting antenna and at least one receiving antenna located in a bed arrangement that supports the patient. In particular, the receiving antenna is located a predetermined distance from a patient reference location to enable detection of electromagnetic energy reflected from a region of the internal organ undergoing asymmetric displacement. The system further includes a radar energizing system that energizes the transmitting and receiving antennas wherein the transmitting antenna irradiates a volume of the patient's body that includes the internal organ. In addition, the receiving antenna detects the reflected electromagnetic energy from the region of the internal organ undergoing asymmetric displacement to enable determination of inhalation and exhalation by the patient.

Those skilled in the art may apply the respective features of the present invention jointly or severally in any combination or sub-combination.

### BRIEF DESCRIPTION OF THE DRAWINGS

The exemplary embodiments of the invention are further described in the following detailed description in conjunction with the accompanying drawings, in which:

FIG. 1 depicts a low gain patch antenna in accordance with an aspect of the invention.

FIG. 2 depicts transmitting and receiving antennas located within a patient bed wherein the receiving antenna is located a distance D from a patient's ear canal.

FIG. 3 includes an upper chart depicting a reflected radar signal that includes a cardiac signal portion and a combined cardiac signal and respiration signal portion and a lower chart depicting I and Q signals, respectively, that correspond to the radar signal.

FIG. 4 shows a simplified block diagram of a radar system in accordance with the invention.

FIG. 5 shows an embodiment of a computed tomography (CT) system that includes the radar system.

To facilitate understanding, identical reference numerals have been used, where possible, to designate identical elements that are common to the figures. The figures are not drawn to scale.

### DETAILED DESCRIPTION

Although various embodiments that incorporate the teachings of the present disclosure have been shown and described in detail herein, those skilled in the art can readily devise many other varied embodiments that still incorporate these teachings. The scope of the disclosure is not limited in its application to the exemplary embodiment details of construction and the arrangement of components set forth in the description or illustrated in the drawings. The disclosure encompasses other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass direct and indirect mountings, connections, supports, and couplings. Further, “connected” and “coupled” are not restricted to physical or mechanical connections or couplings.

The entire disclosure of U.S. patent application Ser. No. 15/972,445, filed May 7, 2018, entitled UHF PHASED ARRAY RADAR FOR INTERNAL ORGAN DETECTION IN A MEDICAL SCANNER by Ahmadreza Ghahremani and James J. Hamill, and that of US Patent Publication No. 2015/0005673 A1 are hereby incorporated by reference in their entirety.

Medical imaging techniques such as positron emission tomography (PET), computed tomography (CT), single-photon emission computed tomography (SPECT) and others are used to obtain images of the interior of a patient's body. During a diagnostic scan utilizing such imaging techniques, the patient's respiratory motion can cause undesirable image artifacts, or the incorrect alignment of two modalities due to internal organ movement that occurs during patient respiration. In order to overcome these disadvantages, it is important to correlate patient inhalation and exhalation with time in a respiration signal so as to provide motion correction of image data.

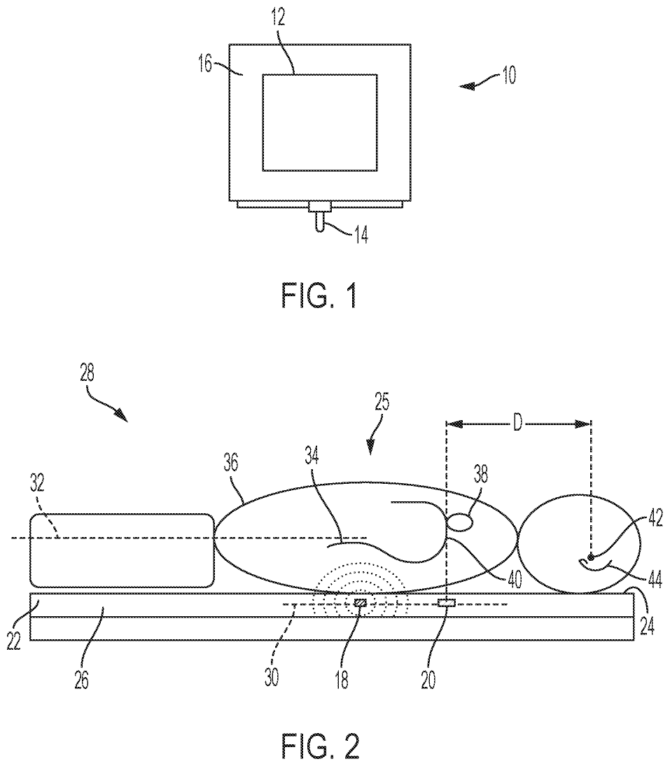

Referring to FIG. 1, a patch antenna 10 in accordance with an aspect of the invention is shown. The antenna 10 is configured for use in a Doppler radar system used to detect a patient's internal organ movement wherein the system operates in the ultra high frequency (UHF) bandwidth of the electromagnetic spectrum. The antenna 10 may be used as either a transmitting or receiving antenna and includes an active layer 12 having a transmission line 14. In an embodiment, the active layer 12 may be fabricated from a metal such as copper. The active layer 12 is located on a substrate 16 fabricated from a material having dielectric properties. In accordance with an aspect of the invention, a dielectric constant for the substrate 16 is sufficiently increased so as to decrease a size of the antenna 10 and reduce antenna gain to form a compact low gain antenna. Reducing antenna gain reduces the volume of the patient's body irradiated by electromagnetic (EM) radiation emitted from a transmitting antenna, thus reducing undesirable reflections of EM radiation received by a receiving antenna from various organs within the body that are not of interest for detecting respiration. Reducing antenna gain also reduces undesirable reflections of EM radiation from surfaces located outside of the patient's body. As a result, undesirable noise in the reflected radar signal is reduced and a signal to noise ratio (SNR) for the antenna 10 is substantially improved. In an embodiment, the substrate 16 has a dielectric constant of approximately 40 and the antenna 10 is configured as a single rectangular patch antenna having an overall size of approximately 2.0 cm×4.2 cm.

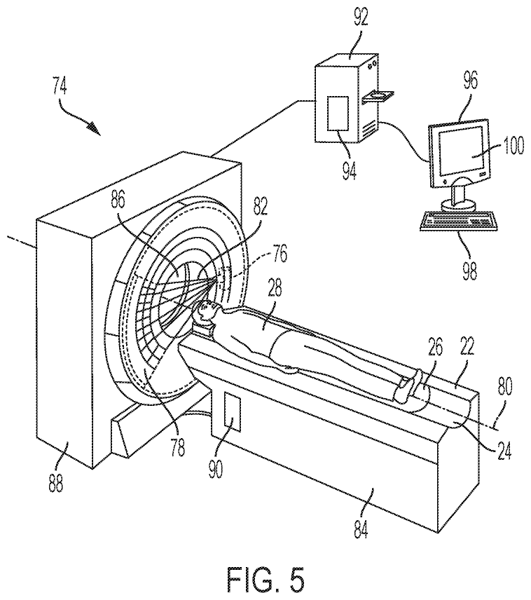

Referring to FIG. 2, transmitting 18 and receiving 20 antennas may be integrated within a patient bed 22. Alternatively, the antennas 18, 20 may be located on a surface 24 of the bed 22. In another embodiment, the antennas 18, 20 may be located within or on a surface of a flexible mat 26 placed on the surface 24 between the patient 28 and the bed 22 (see FIG. 5). Alternatively, the flexible mat 26 may be placed on a top portion of the patient 28. In an aspect of the invention, the antennas 18, 20 are arranged along an antenna axis 30 substantially parallel to a longitudinal axis 32 of the patient 28 (i.e. an axis 32 of the patient 28 extending in a direction between inferior and superior parts of the patient 28) suitable for detecting asymmetric movement of a thoracic diaphragm 34 in a patient's body 36. Alternatively, the antennas 18, 20 may be located on an axis substantially transverse to the longitudinal axis 32. In another embodiment, the antennas 18, 20 are offset relative to each other. Further, an array of transmitting 18 and receiving antennas 20 may be used. For example, the transmitting antennas 18 may be grouped separately from the receiving antennas 20 in the array. Alternatively, the transmitting 18 and receiving 20 antennas may be arranged in pairs in the array. As previously described, the antennas 18, 20 of the invention have a reduced size, thus enabling the antennas 18, 20 to be located relatively close to each other.

The transmitting antenna 18 is located such that the diaphragm 34 is irradiated by EM radiation emitted from the transmitting antenna 18. In an embodiment, the transmitting antenna 18 may be located approximately near a midsection 25 of the patient 28. Due to its proximity to the diaphragm 34, the patient's heart 38 is also irradiated. The diaphragm 34 and heart 38 are structurally more dense and have a higher dielectric constant than nearby organs. Thus, the reflection of EM radiation from the diaphragm 34 and heart 38 is stronger than that from the other organs having a relatively low dielectric constant such as the lung. This facilitates detection of diaphragm and heart movement.

Movement or displacement of the diaphragm 34 is indicative of patient respiration. In addition an object undergoing symmetric movement results in reflected EM radiation that generates a periodic radar signal. It is difficult to determine whether a selected portion of the periodic signal corresponds to either inhalation or exhalation by the patient 28. In accordance with an aspect of the invention, the receiving antenna 20 is located on the bed 22 relative to the diaphragm 34 to enable detection of EM radiation reflected from a portion of the diaphragm 34 undergoing asymmetric movement. Inhalation and exhalation by the patient can then be readily determined from the reflected EM radiation detected by the receiving antenna. In an embodiment, asymmetric movement occurs in an upper region of the diaphragm 34 (i.e. a tip 40 of diaphragm 34) wherein the diaphragm 34 expands and contracts asymmetrically in three dimensional space. Thus, the detection of EM radiation reflected from the diaphragm tip 40 enables determination of patient inhalation and exhalation. It is understood that other areas of the diaphragm 34 that undergo asymmetric movement may be used.

A study was conducted to determine a location on the bed 22 for the receiving antenna 20 (i.e. low gain receiving antenna 20) suitable for detecting asymmetric movement of the diaphragm 34. In the study, a distance D between an ear canal 42 (i.e. a patient reference location) in the patient's ear 44 and the diaphragm tip 40 was measured in topogram images obtained for a plurality of adult patients. As a result of the study, it was determined that the average distance between the ear canal 42 and the diaphragm tip 40 (for adult patients) is approximately 31.7 cm. In accordance with an aspect of the invention, a location for the receiving antenna 20 on the bed 22 suitable for detecting asymmetric movement of the diaphragm 34 is approximately 31.7 cm from the ear canal 42. It is understood that other statistical measures may be used to locate the receiving antenna 20. In addition, physical features of the patient other than, or in addition to, the ear canal 42 may be used as a patient reference location.

In order to optimize placement of the receiving antenna 20 relative to the diaphragm tip 40, an additional approach may be used wherein a cardiac signal is also detected while measuring a respiration signal of the patient. It is known that the heart 38 and diaphragm tip 40 are located relatively close to each other in the human body. Thus, placement of the receiving antenna 20 may be adjusted based on the detected cardiac signal.

### Test Results

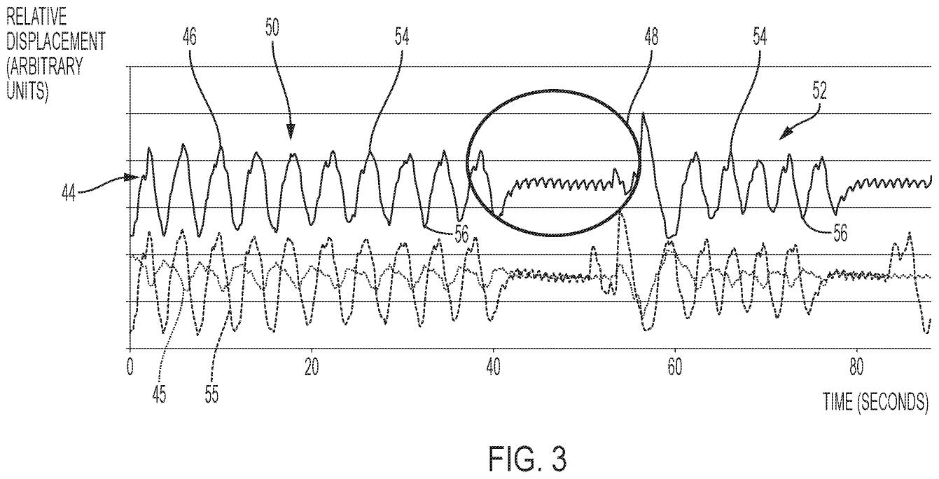

A test was conducted to detect radar signals reflected from internal organs in a patient's body. As part of a test setup, low gain antennas 18, 20 of the invention were configured for use in a Doppler radar system as previously described. The receiving antenna 20 was located on the patient bed approximately 31.7 cm from the patient's ear canal 42 and thus positioned to detect asymmetric movement of the diaphragm 34. Further, the transmitting antenna 18 is configured as a linear polarized antenna and the receiving antenna 20 is configured as a circular polarized antenna, although it is understood that both antennas 18, 20 may be configured as circular polarized antennas in order to improve the SNR. A first chart 44 of a reflected radar signal 46 detected during the test is shown in an upper portion of FIG. 3. During a part of the test, the patient held their breath to eliminate or substantially reduce the effect of respiration on the detected radar signal 46. The part of the test wherein the patient held their breath is shown in circled first area 48 of the first chart 44. The first area 48 depicts a smaller displacement than the remaining portions (i.e. second 50 and third 52 areas) of the radar signal 46. Thus, it can be deduced that the first area 48 represents a cardiac signal and that both the second 50 and third 52 areas include the effect of both the cardiac signal and a detected respiration signal. In addition, the frequency of the signal in the first area 48 is substantially similar to a known cardiac signal frequency, thus further indicating that first area 48 depicts a cardiac signal. Since the receiving antenna 20 is positioned to detect asymmetric diaphragm movement, local maxima 54 and minima 56 of the second 50 and third 52 areas represent patient inhalation and exhalation, respectively. The test results with respect to the respiration signal shown in FIG. 3 were corroborated by a first test utilizing a conventional respiration-correlated gating technique and a second test utilizing the phased array radar system described in U.S. patent application Ser. No. 15/972,445, filed May 7, 2018, entitled UHF PHASED ARRAY RADAR FOR INTERNAL ORGAN DETECTION IN A MEDICAL SCANNER by Ahmadreza Ghahremani and James J. Hamill, the inventors herein. A lower portion of FIG. 3 also depicts second 45 and third 55 charts of the I and Q signals, respectively, that correspond to the radar signal 46.

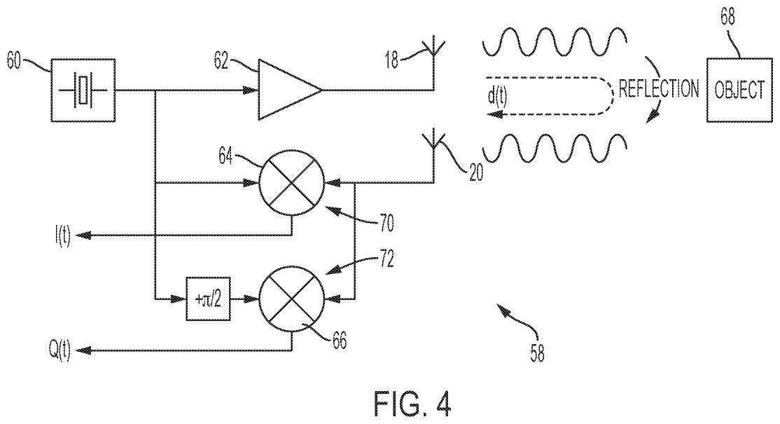

Referring to FIG. 4, a simplified block diagram of a radar system 58 in accordance with the invention is shown. The system 58 includes an oscillator 60, a power amplifier 62 and first 64 and second 66 I/Q mixers. After amplification by the power amplifier 62, a signal is emitted from the transmitting antenna 18 toward an object 68 such as a diaphragm tip 40 of a patient 28. Radio waves reflected from the object 68 are then received by receiving antenna 20. The resulting signal is mixed with the transmitted signal using the first 64 and second 66 I/Q mixers. As the two signals have the same frequency, the mixing result is the phase difference between the signals. The magnitude of the output signals is the magnitude of the received signal minus a mixer conversion loss. The system 58 has two output channels denoted as I(t) and Q(t), the signals of which correspond to:

I(t)=Vᵢ+A cos(φ(t)+φ₀) Eqn. (1)

Q(t)=V_{q}+A sin(φ(t)+φ₀) Eqn. (2)

wherein I(t) is a reference signal, Q(t) is the signal shifted by 90 degrees, Vᵢ, V_{q}, and φ₀ denote constant offsets that are caused by parasitic effects such as antenna crosstalk or nonlinear behavior of the first 64 and second 66 I/Q mixers, A denotes the amplitude of the signal and φ(t) is the phase shift between transmitted and received signals. The phase shift φ(t) is proportional to the distance d(t) from the transmitting antenna to a reflection point on the object 68 and back to the receiving antenna 20. A receiving unit have first 70 and second 72 channels is used in the system 58 to be still able to measure motion if one channel is in a so-called null point. This occurs if the mean distance between the object 68 and the antennas 18, 20 results in a phase shift near to an even multiple of π/2, where small changes of d(t) yield to I(t)=Vi=constant. To overcome this circumstance, the second mixer 66 of the second channel 72 receives an input signal from the oscillator 60 that includes a phase shift of π/2, so that its output is a sine function, as set forth in Eqn. (2). Thus, if one channel is in a null point, the other channel will be in an optimum point.

The invention may be used in conjunction with any type of medical scanning or imaging systems such as positron emission tomography (PET), single-photon emission computed tomography (SPECT), computed tomography (CT), PET/CT systems or radiotherapy systems. For purposes of illustration, the invention will be described in conjunction with a CT system 74 as shown in FIG. 5. The CT system 74 includes a recording unit, comprising an X-ray source 76 and an X-ray detector 78. The recording unit rotates about a longitudinal axis 80 during the recording of a tomographic image, and the X-ray source 76 emits X-rays 82 during a spiral recording. While an image is being recorded the patient 28 lies on the bed 22. The bed 22 is connected to a table base 84 such that it supports the bed 22 bearing the patient 28. The bed 22 is designed to move the patient 28 along a recording direction through an opening 86 of a CT gantry 88 of the CT system 74. As previously described, the transmitting 18 and receiving 20 antennas of the inventive radar system 58 may be integrated within the bed 22. Alternatively, the antennas 18, 20 may be located on a surface 24 of the bed 22. In another embodiment, the antennas 18, 20 may be located within or on a surface of a flexible mat 26 placed on the surface 24 between the patient 28 and the bed 22. Alternatively, the flexible mat 26 may be placed on a top portion of the patient 28.

The table base 84 includes a control unit 90 connected to a computer 92 to exchange data. The control unit 90 can actuate the system 58 (FIG. 4) and the transmitting 18 and receiving 20 antennas. In the example shown here the medical diagnostic or therapeutic unit is designed in the form of a CT system 74 by a determination unit 94 in the form of a stored computer program that can be executed on the computer 92. The computer 92 is connected to an output unit 96 and an input unit 98. The output unit 96 is for example one (or more) LCD, plasma or OLED screen(s). An output 100 on the output unit 96 comprises for example a graphical user interface for actuating the individual units of the CT system 74 and the control unit 90. Furthermore, different views of the recorded data can be displayed on the output unit 96. The input unit 98 is for example a keyboard, mouse, touch screen or a microphone for speech input.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are within the scope of this disclosure.

We claim:

1. A compact radar system for detecting displacement of an internal organ of a patient in a medical scanner, comprising:
at least one transmitting antenna and at least one receiving antenna located underneath the patient, wherein the receiving antenna is located a predetermined distance from a patient reference location to enable detection of asymmetric displacement of the internal organ; and
a radar energizing system that energizes the transmitting and receiving antennas wherein the transmitting antenna irradiates a volume of the patient's body that includes the internal organ and the receiving antenna detects the asymmetric displacement of the internal organ to enable determination of inhalation and exhalation by the patient.

2. The system according to claim 1, wherein the internal organ is a thoracic diaphragm and the asymmetric displacement occurs at a tip of the thoracic diaphragm.

3. The system according to claim 1, wherein the predetermined distance is determined statistically based on measurements of a distance between the patient reference location and a tip of a thoracic diaphragm in a plurality of patients.

4. The system according to claim 3, wherein a cardiac signal is detected simultaneously with a respiration signal of the patient wherein the detected cardiac signal is used to optimize placement of the receiving antenna relative to the tip of the thoracic diaphragm.

5. The system according to claim 1, wherein the patient reference location is an ear canal of the patient.

6. The system according to claim 1, wherein a substrate for each antenna has a relatively high dielectric constant to provide compact low gain antennas.

7. The system according to claim 1, wherein the transmitting and receiving antennas are located in a mat positioned underneath the patient.

8. A compact radar system for detecting displacement of an internal organ of a patient in a medical scanner, comprising:
at least one transmitting antenna and at least one receiving antenna located in a bed arrangement that supports the patient, wherein the receiving antenna is located a predetermined distance from a patient reference location to enable detection of electromagnetic energy reflected from a region of the internal organ undergoing asymmetric displacement; and
a radar energizing system that energizes the transmitting and receiving antennas wherein the transmitting antenna irradiates a volume of the patient's body that includes the internal organ and the receiving antenna detects the reflected electromagnetic energy from the region of the internal organ undergoing asymmetric displacement to enable determination of inhalation and exhalation by the patient.

9. The system according to claim 8, wherein the internal organ is a thoracic diaphragm and the asymmetric displacement occurs at a tip of the thoracic diaphragm.

10. The system according to claim 8, wherein the predetermined distance is determined statistically based on measurements of a distance between the patient reference location and a tip of a thoracic diaphragm in a plurality of patients.

11. The system according to claim 10, wherein a cardiac signal is detected simultaneously with a respiration signal of the patient wherein the detected cardiac signal is used to optimize placement of the receiving antenna relative to the tip of the thoracic diaphragm.

12. The system according to claim 8, wherein the patient reference location is an ear canal of the patient.

13. The system according to claim 8, wherein a substrate for each antenna has a relatively high dielectric constant to provide compact low gain antennas.

14. The system according to claim 8, wherein the bed arrangement includes a mat that includes the transmitting and receiving antennas.

15. A method for locating a receiving antenna in a radar system that detects displacement of an internal organ of a patient in a medical scanner, comprising:
measuring a distance between a patient reference location and a region of the internal organ that moves asymmetrically, wherein the distance is measured in a plurality of patients to provide a plurality of measured distances;
calculating a statistical measure for the measured distances to determine a calculated distance; and
locating the transmitting antenna in a patient bed wherein the transmitting antenna is spaced apart from the patient reference location by the calculated distance.

16. The method according to claim 15, wherein the internal organ is a thoracic diaphragm and the asymmetric displacement occurs at a tip of the thoracic diaphragm.

17. The method according to claim 16, further including detecting a cardiac signal simultaneously with a respiration signal of the patient wherein the detected cardiac signal is used to optimize placement of the receiving antenna relative to the tip of the thoracic diaphragm.

18. The method according to claim 15, wherein the patient reference location is an ear canal of the patient.

19. The method according to claim 15, further including positioning a mat that includes the transmitting and receiving antennas between the patient and the patient bed.

20. The method according to claim 15, wherein a substrate for each antenna has a relatively high dielectric constant to provide compact low gain antennas.
